(19) United States
(12) Patent Application Publication
(10) Pub. No.: US 2013/0085357 A1
(43) Pub. Date: Apr. 4, 2013

(54) HAEMODYNAMIC MONITORING DEVICE

(71) Applicants: Wolfgang Huber, Gmund (DE); Stephan Joeken, Schopfheim (DE); Martin Peterreins, Sauerlach (DE)

(72) Inventors: Wolfgang Huber, Gmund (DE); Stephan Joeken, Schopfheim (DE); Martin Peterreins, Sauerlach (DE)

(21) Appl. No.: 13/628,922

(22) Filed: Sep. 27, 2012

(30) Foreign Application Priority Data
Sep. 30, 2011 (DE) 102011114666.4

(51) Int. Cl. A61B 5/0205; A61B 5/00; A61B 5/024; A61B 5/145; A61B 5/021; A61B 5/0402; A61B 5/01; A61B 5/02; A61B 5/029

(52) U.S. Cl. CPC A61B 5/0205; A61B 5/02; A61B 5/7278; A61B 5/024; A61B 5/029; A61B 5/021; A61B 5/0402; A61B 5/01; A61B 5/02055; A61B 5/14542
USPC 600/364; 600/481; 600/508; 600/500; 600/526; 600/485; 600/509; 600/513; 600/484; 600/483

(57) ABSTRACT

A relation is formed between an n-tuple having n components and formed at a first point in time and at least one other n-tuple having n components formed at at least one corresponding later point in time, wherein n is a natural number equal to or greater than 1, and the components comprise at least one derived parameter and/or one read-in data value. If this relationship satisfies a predetermined calibration criterion, a calibration signal is triggered and is displayed, and/or automatically triggers a recalibration of the haemodynamic monitoring device. For example, the pulse contour cardiac output PCCO is derived from the arterial pressure curve as the constituent component of a 1-tuple. As long as this differs from the reference cardiac output CO_{Ref} by less than a predefined threshold value, for example 101 or 15% of the reference cardiac output, parameter determination continues without initiating a new calibration. On the other hand, if the deviation exceeds PCCO-CO_{ref} I, the calibration signal is triggered.

This application claims the benefit of German Patent Application No. ID 2011 114 666.4, filed Sep. 30, 2011, the entire contents of which are incorporated herein by reference.

### TECHNICAL FIELD

The present invention relates generally to haemodynamic monitoring of patients. In particular, the present invention relates to a haemodynamic monitoring device with reading in means for repeated reading in of data representing at least one physical variable, calculation means for calculating at least one parameter from the read-in data, and calibration means for calibrating the device.

### PRIOR ART

Various systems used for haemodynamic monitoring of a patient are known, wherein a distinction is drawn in the clinical context between basic haemodynamic monitoring (including electrocardiography (EKG), pulse oximetry, continuous or intermittent blood pressure measurement and central venous pressure (CVP) measurement) and extended haemodynamic monitoring. Extended, haemodynamic monitoring also includes recording and determining global, cardiovascular parameters such as cardiac output (CO), central venous oxygen saturation (ScvO2) and the cardiac preload and afterload among others. In methodological terms, the procedures for oximetry, pulse contour cardiac output (PCCO), transoesophageal echocardiography, pulmonary artery catheterisation are available, as well as cardiac output determination using various indicator dilution methods (transpulmonary thermodilution, lithium dilution and non-invasive (for example CO₂-based) methods).

Although in principle the invention is not subject to any restrictions regarding its application in various systems used for haemodynamic monitoring of a patient, the invention particularly relates to the calibration or recalibration of patient monitors as part of a haemodynamic monitoring procedure with pressure and/or temperature measurement, for example by pulse contour analysis and/or thermodilution measurement, preferably employing catheters. Such patient monitors are known in various embodiments from the prior art. Pressure and/or temperature measurements employing catheters are used in determining haemodynamic parameters such as mean arterial pressure (MAP), cardiac output (CO) global end diastolic volume (GEDV) and extravascular lung water (EVLW).

By analysing the pulse contour that is recorded via arterial pressure measurement, it is possible to determine the stroke volume (SV) for each heartbeat by using mathematical methods to calculate the stroke volume of the heart from the course (the “shape” or waveform) of a blood pressure curve that is measured continuously in a leg artery, for example. In these circumstances, the pressure that is measured in the peripheral artery is approximately equivalent to the aortic pressure. The basis of the mathematical methods is the extraction and clinically usable representation of the information contained in the arterial blood pressure curve. The cardiac output is calculated from the area below the pressure curve that is above the diastolic pressure and corresponds to the period of time for which the aortic flap is open. In this way, the pulse contour analysis may be used to determine the changes in stroke volume (stroke volume variation SVV) caused by-respiration over the breathing cycle, wherein the SVV occurs as a result of a fluctuating preload and accordingly may be used in an estimation of the volume responsiveness of the left ventricle.

The determination of cardiac output (CO) using pulse contour analysis on the basis of a non-linear Windkessel model is described in EP 0 347 941 B1.

The pulse contour analysis system is usually calibrated by indicator dilution or thermodilution. Most of the different commercially available thermodilution systems work with a cold indicator, that is to say a cooled bolus. In transpulmonary thermodilution measurement, a defined quantity of cold liquid is injected into the vein of a test-subject and the transpulmonary development of the blood temperature is recorded using a thermal probe placed in a peripheral artery (for example the femoral or radial artery). Measurement of the temperature in thermodilution procedures is usually carried out with a thermistor, that is to say a resistance temperature detector (RTD). The use of RTDs is widespread due to their stability and their high degree of accuracy, and they have an almost entirely linear measurement, signal.

Methods and devices for transpulmonary thermodilution measurement are disclosed in U.S. Pat. No. 5,526,817 and U.S. Pat. No. 6,394,961 and elsewhere. In U.S. Pat. No. 5,526,817, a method for determining circulatory fill status by thermodilution is described, wherein various volumes and volume flows are determined, particularly the global end diastolic volume (GDEV), the intrathoracic blood volume (ITBV), the pulmonary blood volume (PBV), extravascular lung water volume (EVLW), the intrathoracic thermo-volume ITTV), the pulmonary thermo-volume (PTV) and the global cardiac function index (CFI), for the purpose of evaluating a patient's circulatory fill status.

As was indicated in the preceding, thermodilution and pulse contour methods are often used in combination, which enables the thermodilution measurement to be included advantageously in the calibration of the pulse contour method. In this case, after the initial calibration 2-3 calibrations per day are declared as the standard recalibration interval in routine clinical practice in order to guarantee sufficiently reliable continuous CO measurement using pulse contour analysis, since the stability of known pulse contour algorithms is not assured under all circumstances. Although the long interval reduces the commitment in terms of resources and staff, possibly also avoiding a volume overload with regard to the thermodilution measurement, on the other hand an excessively long interval between recalibrations can result in clinical evaluation errors, since changes that occur during the interval, such as variations in the clinical situation, catheter dislocation or arrhythmias do not cause the system to be recalibrated.

### BRIEF DESCRIPTION OF THE INVENTION

Based on the devices and methods known from the related art, the underlying object of the present invention is to provide a device and method for haemodynamic monitoring of a patient that either eliminate or significantly diminish the drawbacks outlined in the preceding.

This object is solved with an automatic or manual calibration or recalibration for haemodynamic monitoring of a patient that is adjusted according to the actual needs.

In a first aspect, the present invention therefore provides a device for haemodynamic monitoring of a patient that comprises reading in means for repeated reading in of data representing at least one physical variable, calculation means for calculating at least one parameter with the aid of the read-in data, and calibrating means for calibrating the device, and is also furnished with triggering means for triggering a calibration signal. In this context, the triggering means are designed to trigger the calibration signal depending on the change over time in the read-in data and/or at least one of the parameters. The present invention thus provides a device for haemodynamic monitoring of a patient that enables a significantly improved recalibration procedure compared with the related art. The recalibration technique according to the invention is also based on the actual changes in the patient, so that recalibration may be reliably initiated when a significant deviation in the overall haemodynamic situation occurs when compared with the patient's previous condition. With the device according to the invention and the corresponding method, unnecessary measurements and unnecessary volume burden, as part of a repeated thermodilution measurement for example, may be avoided as well as unnecessary additional use of resources and staff. Devices and methods according to the invention may also provide greater validity of information with regard to real changes.

In a preferred implementation, the triggering means may be designed to determine an n-tuple having n components that incorporate at least one parameter and/or value of the read-in data, n being an integer of 1 or more. Tuples with n>1 components (n>1 dimensions) are used preferably. The triggering means may also form a relation, preferably an arithmetical relation, between an n-tuple determined at a first point in time and at least one n-tuple determined at least one later point in time, in which each n-tuple has n components, via a univariate or multivariate analysis procedure. Particularly in the case of tuples with n>1 dimensions, multivariate analysis procedures may be employed to advantage. However, univariate procedures may also be used with corresponding data reduction or weighting. A difference may preferably be determined between the n-tuple that is determined at a first point in time and at least one n-tuple that is determined at at least one later point in time. In general, a difference may be determined, for example as part of a determination of a progression over a period of time defined by multiple points in time, and the difference may preferably be determined between a first and a second point in time.

In another preferred embodiment the triggering means are designed to carry out a mathematical classification method. Preferably, the classification method may be implemented in the form of a support vector machine, or it may be a discriminant analysis. A discriminant analysis is understood to be a method of multivariate statistical procedures that is used to differentiate between two or more groups. The groups are described with a plurality of features (e.g. also variables). In the device according to the invention, the significance of discriminant variable Yₘ derived from the discriminant analysis may be determined by the triggering means. If the discriminant variable Yₘ derived from a first and from a subsequent second determined differs by a predefined deviation value from discriminant variable Y_{Ka1}, which was determined at a temporally close interval to a calibration, a calibration signal may be triggered by the triggering means. In this context, deviation values may be absolute or defined as a percentage of a previously established value. An advantageous deviation value is a deviation by 5% to 15% of the most recently determined discriminant variable Yₘ from discriminant variable Y_{Ka1}. Alternatively, discriminant variables Yₘ and Yₘ₊₁, the latter determined directly after the former, may be correlated by discriminant analysis.

An n-tuple with only n−1 or n−(>1) components may be determined, preferably by thermodilution, before the initial calibration, since no parameters and/or variables are (yet) available from a previous calibration. If discriminant variable Y_{Ka1} derived from a first and a subsequent, second determination by the triggering means differs from a predefined reference variable Y_{R} by a predefined deviation value, a calibration signal may be triggered, wherein reference variable Y_{R} may be a (particularly initially) measured variable or a comparison value (stored or entered beforehand) that has been predetermined in some other way. In this case, the n-tuple that has been reduced by n−1 or n−(>1) may also serve as an indicator for a system change.

In a further advantageous embodiment of the device according to the invention, the triggering means may be designed to perform a linear discriminant analysis. Although in general for example the variants of square, regularised, diagonal or nearest-centroid discriminant analysis may also be applied, linear discriminant analysis has the advantage that it is easy to carry out with only low model complexity. In addition, normally distributed datasets with largely corresponding covariance matrices may be advantageously included for the purposes of linear discriminant analysis. This particularly enables the decision limits for used datasets (recalibration yes/no) to be defined precisely.

The n-tuple with n components may preferably include at least one parameter selected from the group of heart rate (HR), pulse duration (T), stroke volume (SV), mean arterial pressure (MAP), pulse contour cardiac output (PCCO), thermodilution cardiac output (CO_{TD}), stroke volume variation (SVV), systemic vascular resistance (SVR), pulse pressure variation (PPV), fluid responsiveness index (FRI), ventricular contractility (for example dPmx), atrial pressures and/or ventricular pressures, (for example RAP, RVEDP, LVEDP), EKG section data and EKG intervals as well as characteristics of the medication that affects haemodynamics and respiratory parameters that affect haemodynamics, Suitable EKG sections and EKG intervals are for example the height and width of the P-wave, of the T-wave, of the QRS complex, and the length of the PQ interval, of the ST segment. Extrasystoles and other factors that disrupt cardiac rhythm may also be considered. Characteristics of the medication that affects haemodynamics may include the dosage of a given medication, for example, the breathing parameters that affect haemodynamics may include the positive end expiratory pressure (PEEP), the average airway pressure or the ventilation mode.

The n-tuple may particularly preferably include at least one parameter with the greatest possible significance, which may preferably be measured and/or determined by means of an existing monitoring device in non- or minimally invasive manner, that is to say as simply as possible. In particular, parameters may be suitable that can be determined without the need for prior calibration and/or whose deviation from a reference and/or prior value is easily detectable. For the purposes of the invention, these include ail cardiovascular parameters that are advantageously determinable without the use of more invasive procedures and the determination of which is subject to only minor systematic errors, such as heart rate, pulse duration, estimated mean arterial pressure, and certain EKG parameters. Parameters that are determined using more complex non-invasive procedures, for example parameters from prolonged echocardiography or impedance cardiography may also serve as components of an n-dimensionality constructed tuple. The pulse contour cardiac output (PCCO), systemic vascular resistance (SVR) and thermodilution cardiac output (CO_{TD}) parameters in particular may be obtained very easily with the aid of the preferred measuring arrangement for haemodynamic monitoring using minimally invasive, catheter-mediated pressure and/or temperature measurement, for example by pulse contour analysis and/or thermodilution measurement.

In a further embodiment of the device according to the invention, the n-tuple with n components may include at least one data value selected from the group of arterial pressure (P), central venous pressure (CVP), blood temperature (Tb), peripheral oxygen saturation (SpO2), central venous oxygen saturation (ScvO2). The n-tuple may particularly preferably include data values having the greatest possible absolute significance, which may be measured and/or determined preferably minimally or non-invasively with the aid of an existing monitoring device, that is to say as simply as possible. In particular, data values that can be measured without the need for prior calibration and/or whose deviation from a reference and/or prior value is easily detectable may be suitable.

In a further embodiment of the device according to the invention, the temporal interval between the first and the at least one temporally later determination of the n-tuple with n components may be less than one hour. In this respect, the preferred temporal interval may vary according to the components of the n-tuple. Particularly with an n-tuple whose components comprise parameter and data values that can be measured non-invasively and/or are easily obtainable (for example HF, SpO2), a short interval period between the individual tuple determinations may be selected. Correspondingly, a longer interval period between the individual tuple determinations may be selected if the components of the tuple contain parameter and data values that are measurable by invasive means and/or are more difficult to obtain (for example ScvO2). A time interval not exceeding 15 minutes down to the period of a few or single heartbeats (beat-to-beat assessment as to whether recalibration is necessary). Particularly with short time intervals, the parameter space may be determined continuously, preferably when using data values and/or parameters that either do not require any initial calibration or that can be measured and/or calculated with little effort using an existing monitoring arrangement.

For facilitating implementation or for avoiding negative side-effects of invasive steps performed too frequently in connection with a certain calibration method, it may also be advantageous to implement a pre-defined minimum time interval between two succeeding calibrations, e.g. ten seconds, 30 seconds, one, five, ten, 15 or 30 minutes, and/or to implement a maximum number of calibrations per unit time, e.g. any between two and twenty per hour. Such minimum time intervals and maximum numbers of calibrations per unit time may vary greatly depending on the circumstances of applying the invention, e.g. on the complexity of the calibration method or the degree of invasiveness of steps performed in combination with the particular calibration method.

It is particularly preferred if the triggering means are designed to take into account (preferably by filtering or averaging) consecutive changes, in the respective other direction, of the read-in data and/or at least one of the parameters which occur temporally within a predetermined interval and which deviate in the same direction from both the parameter derived at the immediately preceding point in time and the parameter derived at the immediately following point in time (“outliers”) in such manner as to reduce the weighting of the data that was read in at a given time and/or of the parameter that was derived at a given time. The weighting may be reduced progressively as far as a point of ignoring an “outlier” altogether by means of a value selection algorithm, but may also be achieved simply for example by averaging over multiple consecutive parameter or data values. This treatment of inconsistent consecutive changes over time in the read-in data and/or at least one of the parameters may be understood descriptively as smoothing of the trend over time of the read-in data or the read-in parameter.

In a further embodiment of the device according to the invention, the calibration signal that is triggered by the triggering means may initiate an automatic calibration or recalibration. The manual calibration or recalibration that is usually carried out in routine clinical procedures may be replaced by a corresponding automatic calibration or recalibration. An automatic calibration or recalibration may thus be carried out particularly advantageously if the calibration process does not require any additional invasive or only a minimally invasive procedure. Bolus injections via an electronically controllable injection pump are conceivable in this context, for example. Alternatively, a thermodilution technique may be used such as is described in EP 1 236 435 A1, which does not require the injection of a cold bolus but instead makes use of a local temperature deviation effected by a heat generator or Peltier cooling element or the like.

In a further embodiment, the triggering of the calibration signal may be indicated visually and/or acoustically, for example by a standard display device or sound generator, wherein the display of the calibration signal preferably extends beyond the display of a value that is shown continuously in any case. That is to say, the display not only changes to reflect the switching of a displayed numerical value, but the triggering of the calibration signal advantageously alerts the user to this change, without the user having to perform this value comparison himself. The mere continuous representation of a measurement value that is being displayed in any case or a parameter derived therefrom and displayed continuously is thus not an output, of a calibration signal for the purposes of this embodiment of the present invention.

When the calibration is initiated automatically, the user is preferably alerted to this by the acoustic or visual signal. According to an alternative advantageous embodiment, however, the user who is alerted by the signal may also initiate the recalibration by manual intervention. The calibration signal then merely signals that it is necessary to perform the calibration or recalibration, thus advantageously leaving the evaluation of the clinical situation to the judgment of the user. It is also possible to implement an advantageous refinement according to which the user may also specify an individually adjusted and variable time interval after which an automatic calibration is to be performed without any further signal from the user.

According to a further advantageous embodiment of the invention, the triggering means may be configured to perform a two or multistage check of the dependency of the change over time in the read-in data and/or at least one of the parameters. In this context, the two- or multistage check comprises the hierarchical check of at least two triggering criteria that cause the triggering of the calibration signal and are dependent on the change over time in the read-in data and/or at least one of the parameters. For example, the triggering means may first check whether a certain parameter or measurement value, has changed by more than a predetermined factor within 3 specified period of time, and if this criterion is met, it may check as a second criterion how uniformly the change took, place over the specified time period. A particularly uniform change may be interpreted for example as flatline drift and trigger the calibration signal. Other hierarchical checks of trigger criteria for the calibration signal are also implementable.

In another preferred embodiment, the device additionally comprises evaluation means for evaluating the arithmetical relationship between data that is read in immediately before and data that is read in immediately after a calibration, and/or between at least one parameter that is determined immediately before and at least one parameter that is determined immediately after a calibration. In this context, immediately may mean the measurement or parameter determination directly previous to or after the calibration, or also another measurement or parameter determination respectively within a time period before and after the calibration, wherein the time period is small, that is to say preferably less than two minutes, particularly preferably less than one minute.

The evaluation means may be used to carry out an evaluation of the recalibration or of the recalibrated haemodynamic parameter. For example, the pulse contour cardiac, output determined immediately before a recalibration and the pulse contour cardiac output determined immediately after a recalibration may be correlated with one another. In this process, the evaluation means may evaluate whether the recalibration that was performed due to the change over time in the read-in data and/or the at least one parameter was necessary or not on the basis of the magnitude of the deviation between the two parameters. If there is no difference or only a minor difference between data that was measured or a parameter that was derived immediately before and after the calibration, this may be evaluated as an indication that the inordinate change in the read-in data and/or the at least one parameter over time has physiological origins and is riot attributable to measurement or calculation inaccuracies.

The evaluation means may also serve to evaluate the data values or parameters in the n-tuple that are taken into account by the triggering means. For example, if haemodynamic parameter in question is unchanged after the recalibration, it may be that the change over time in the data value or the at least one parameter that is considered by the triggering means is not suitable for correctly indicating a change in the patient's situation with regard to the haemodynamic parameters of interest. Thus, the evaluation carried out by the evaluation means may also serve appropriately for performing a corresponding evaluation of the parameter and data space of the n-tuple, and modifying it as necessary. This evaluation and/or modification may also be performed automatically for example via corresponding algorithms and may result in a “learning process” of the device (machine learning). An n-tuple consisting of n components may be modified on the basis of the results of the evaluation by applying different weighting to individual components, for example, or components may be added or omitted.

In another preferred embodiment, the device further comprises input means for entering specific information about the patient. This specific information about the patient may contain categorising information and/or bioraetric information. In this way it is possible to include bioraetric data in the determination of reference values particularly during the initial calibration. But the use of bioraetric data is also advantageous for forming n-tuples having n>1 components, since they allow read-in data values and parameters to be indexed, and the statistical and/or clinical significance of the data values and parameters indexed in this way is increased. A calibration criterion depending on entered information may also be established. Depending on whether the patient belongs to a category that prompts the expectation of a given parameter change or hot (possibly because the patient is undergoing a certain course of treatment during the haemodynamic monitoring), the triggering means may attach more or less importance to this parameter or measurement value when evaluating whether a calibration signal is to be triggered or not. In other words, this enables changes in the parameter and measurement values that are to be expected to be excluded from the evaluation as to whether recalibration is necessary. For example, if a patient has received antipyretic medication, it may be reasonable to ignore the measured blood temperature in an evaluation algorithm that normally considers the blood temperature as an input value for triggering a calibration signal.

In a further aspect, the present invention relates to a method for monitoring a patient's haemodynamic condition that comprises the reading in of data representing at least one physical variable, calculating at least one parameter with the aid of the read-in data, triggering a calibration signal in response to the change over time in the read-in data and/or at least one of the parameters, and (re)calibrating the device.

According to an advantageous embodiment, the method also comprises an evaluation of the arithmetical relationship between the read-in data and/or at least one of the parameters determined immediately before and one of the parameters determined immediately following a (re)calibration.

In general, any variant of the invention described or implied as part of the present application may be particularly advantageous, depending on the economical and technical circumstances in each individual case. Individual features of the embodiments described may be substituted and used with each other in any combination unless otherwise indicated, and provided such substitution or combination is fundamentally technically feasible.

### BRIEF DESCRIPTION OF THE DRAWING

In the following, several particularly advantageous embodiments of the invention will be described in non-exhaustive, non-limiting manner for exemplary purposes with reference to the accompanying drawings. The particular embodiments are intended particularly to explain the inventive idea, but the invention is not limited solely to the aspects represented herein.

FIG. 1 is a schematic view of the cardiovascular system of a patient undergoing haemodynamic monitoring with a preferred embodiment of the device according to the invention.

FIG. 2 shows the flow diagram, of a calibration signal triggering process according to the invention in generalised form for several possible embodiments.

FIG. 3 shows a flow diagram for the calibration signal triggering process according to an embodiment with the change over time of a single parameter of the calibration criterion.

FIG. 4 shows a flow diagram for the calibration signal triggering process according to another embodiment with two hierarchically applied calibration criteria.

FIG. 5 shows a flow diagram for the evaluation of the calibration result according to another preferred embodiment of the invention.

### DETAILED DESCRIPTION OF PREFERRED EMBODIMENTS

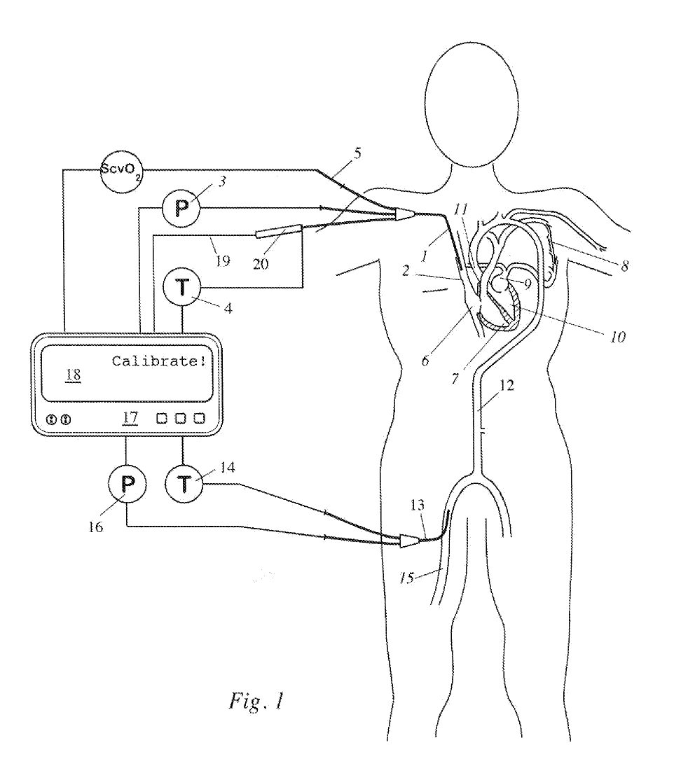

FIG. 1 is a schematic representation of the main components of a preferred embodiment of the device according to the invention. A multi-lumen central venous catheter 1 is positioned in the vena cava superior 2 of a patient. A pressure measurement line 3 with a pressure sensor known per se from the prior art is used, for continuous or intermittent measurement of the central venous pressure. Central venous catheter 1 is also furnished with an injection channel 20 with injectate temperature sensor 4. Central venous catheter 1 is also equipped with an optical measuring probe 5 for measuring the central venous oxygen saturation.

A bolus of fluid (for example 10 ml or 0.15 ml/kg) serving as a temperature indicator and being either significantly warmer or significantly colder than the temperature of the patient's blood is injected into central venous catheter 1 for a thermodilution measurement. The bolus induces a localised temperature deviation in the patient's bloodstream. This temperature deviation migrates from the injection site first to the right atrium 6 and ventricle 7, then follows the pulmonary circulation system 8, passes through the left atrium 9 and the left ventricle 10, and finally reaches the systemic circulation 12 via the aorta 11. A temperature sensor 14, which is arranged in a peripherally localised arterial catheter 13, measures the local temperature deviation as a function of time. Any migrating temperature deviation may be measured in that way, as a response by the system to a defined input signal. The local blood temperature may also be measured continuously via temperature sensor 14. The measurement site is preferably a peripheral artery 15, such as (as shown) the A. femoralis, A. radialis or A. axillaris. Arterial catheter 13 is also provided with a pressure measurement line including a pressure sensor 16, known per se from the prior art, which measures the local pressure in the peripheral artery 15 as a function of time.

The sensors of pressure lines 3, 16 and temperature sensors 4, 15 are connected to a computer system 17 for the purpose of transferring the input signals, the corresponding system responses, and the pressure and temperature signals to the memory unit of computer system 17, so that this measured data is then available for further processing. Computer system 17 is equipped with a corresponding executable program that reads in the data values and is capable of determining a wide range of haemodynamic parameters from the data values. The calculation means according to the invention are thus implemented using suitable program routines. Thus particularly, the temperature measured by arterial temperature sensor 14 is recorded as a thermodilution curve that corresponds to the system response to the bolus injection through injection channel 11. From thermodilution curve 18, computer system 17 is able to calculate the thermodilution-based cardiac output (CO_{TD}) using known algorithms such as the Steward-Hamilton equation. The thermodilution measurement may also serve as the basis for determining parameters such as the global end diastolic volume (GEDV), the intrathoracic blood volume (ITBV) and the extravascular thermovolume (ETV) in known manner on computer system 17.

The pulse contour cardiac output (PCCO) as well as the stroke volume (SV) and the stroke volume variation (SVV) may be determined using known algorithms from the arterial pressure curve recorded via the sensor in, arterial pressure line 16. The PCCO may be calibrated using the reference CO_{TD} that is determined by thermodilution measurement.

Computer system 17 is also connected to a medicinal dosing device in injection channel 20 via a control channel 19. The medicinal dosing device may serve as the means for carrying out the automatic bolus injection for the thermodilution measurement, or an injection channel for performing manual bolus injections may be provided. As part of an automatic calibration routine, computer system 17 sends a signal to the medicinal dosing device via control channel 19, upon which a bolus of fluid is injected through central venous catheter 1 for (re)calibration. Read-in data values and/or calculated parameters as well as signals can be viewed by the user on the display 18 integrated in computer system 17.

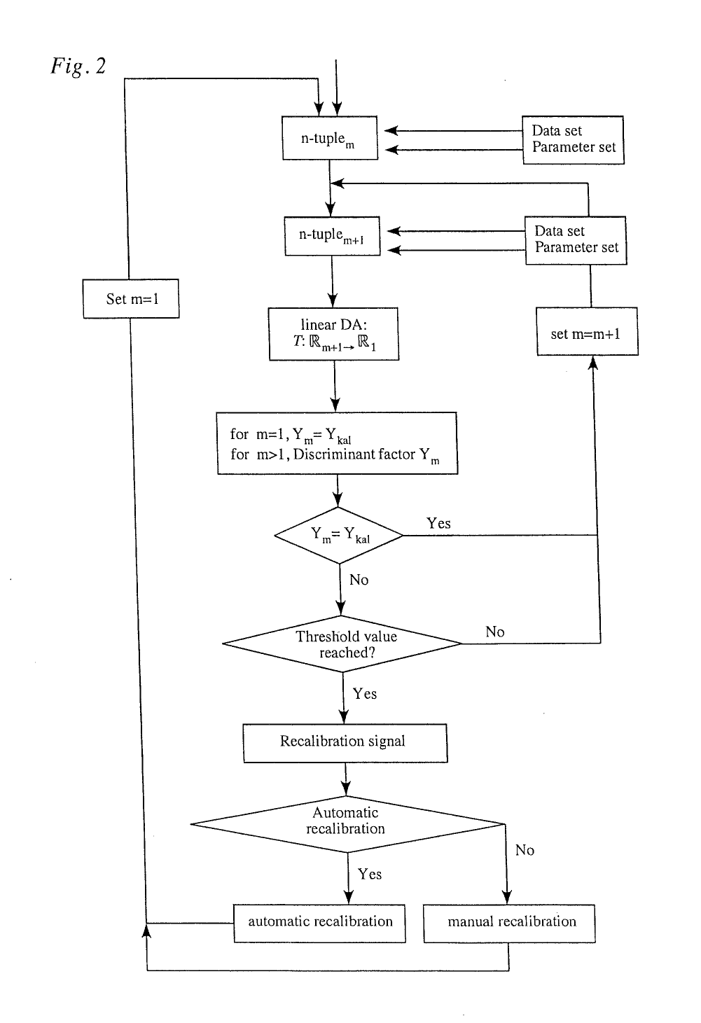

FIG. 2 shows a generalised flow diagram of the initiation of a recalibration according to the invention. The data values read in via the read-in means integrated in computer system 17 and/or the parameters calculated by the calculation means integrated in computer system 17 are merged by the triggering means that are also integrated in computer system 17 at a first point in time m in a first n-tuple having n components and forming n-tupleₘ. A second n-tuple is determined at a later point in time, for example 5 minutes later, using the data values read in by the read-in means and/or the parameters calculated by the calculation means, and this forms n-tupleₘ₊₁. The discriminant factor Yₘ is derived from the linear discriminant analysis carried out by the triggering means in the next step. The first discriminant factor Yₘ derived after a calibration from n-tuple₁ and n-tuple₂ corresponds to the reference discriminant factor Yₖₐ₁. Discriminant factor Yₘ is derived by linear discriminant analysis performed in the next step by the triggering means from the n-tupleₘ₊₁₊₁ (that is to say n-tuple₃) and n-tuple₁ at another, later point in time. In the next step, discriminant factor Yₘ is then correlated with reference discriminant factor Y_{Ka1} for example in the form of a simple comparison. If the two values are the same, that is to say Yₘ=Y_{Ka1}=“yes”, another n-tuple (that is to say n-tuple₄) is determined by the triggering means at a subsequent point in time (m=3+1) and another linear discriminant analysis is performed. If the two values are not the same, that is to say Yₘ=Y_{Ka1}=“no”, the difference between Yₘ and Y_{Ka1} is determined for example as a percentage or an absolute value, and in a next step a comparison is made as whether a predefined, relative or absolute threshold value has been reached. Accordingly, a difference of for example 15% between the last determined discriminant variable Y₃ and Y_{Ka1} causes a calibration signal to be triggered by triggering means, because the threshold deviation value of 15% has been reached (“yes”). On the other hand, if the difference between the most recently determined discriminant variable Y₃ and Y_{Ka1} is smaller, only 1% for example, a calibration signal is not triggered by the triggering (“no”), another n-tuple is determined instead. If a calibration signal has been triggered by the triggering means, this being shown to the user in the display with the message “Calibrate!”, in a next step it is determined whether an automatic recalibration should be carried out. Automatic recalibration may be set by the user before beginning haemodynamic monitoring, or it may also be switched from manual to automatic recalibration while a haemodynamic monitoring procedure is in progress. After a recalibration, the counter values (m) of the n-tuples are reset to the starting state (m=1). All n-tuples and discriminant variables that are determined by the triggering system between two calibrations are stored by the memory unit in computer system 17.

The automatic recalibration option is not available if, other than in the arrangement shown in FIG. 1, no bolus injection means that are controllable by computer system 17 are provided. In such a configuration of the invention, the user has to initiate the calibration manually when the display indicates that the calibration signal has been triggered.

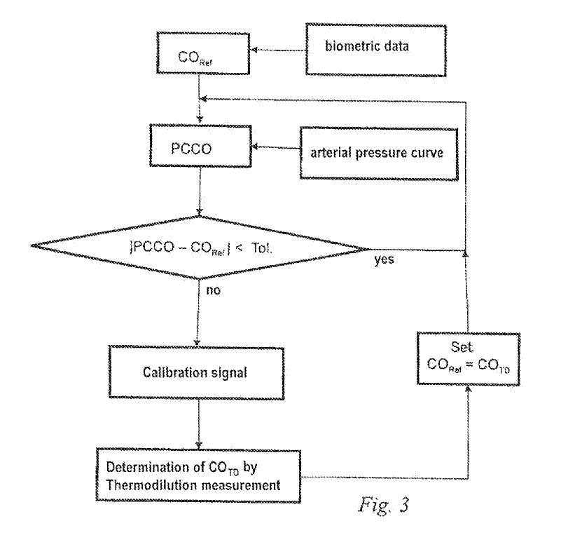

FIG. 3 shows a flow diagram for the initiation according to the invention of a recalibration using the example of a 1-tuple with the pulse contour cardiac output (PCCO) as the constituent component. First, specific information about the patient is entered, for example via a display 18 designed for touchscreen entry, wherein the specific information includes (for example sex and age) and biometric (for example height and weight) information and is used to create the minimal cardiac output as a reference cardiac output CO_{Ref}. Alternatively, a reference cardiac output may also be determined by thermodilution measurement.

The pulse contour cardiac output PCCO is determined from the arterial pressure curve. As long as this differs from the reference cardiac output by less than a predefined threshold value (tolerance “Tol”), for example 10% or 15% of the reference cardiac output, parameter determination continues without, recalibration. On the other hand, if the difference |PCCO−CO_{Ref}| exceeds the threshold value, a calibration signal is triggered.

It may also be advantageous to define different threshold, values for an upward deviation and a downward deviation, that is to say the criterion for triggering a calibration signal may be dependent on the difference being positive or negative, that is to say calibration will not take place as long as Toll <PCCO−CO_{Ref}|<Tol2. According to one configuration selected purely for exemplary purposes, a calibration signal may be triggered when the difference PCCO−CO_{Ref} is no longer in the range

−0.1 CO_{Ref}<PCCO−CO_{Ref}<0.15 CO_{Ref}

The triggering of a calibration signal may be displayed in order to call a manual calibration, or if the requisite equipment is present, as shown in FIG. 1, trigger an automatic calibration. The cardiac output CO_{TD} determined in the calibration measurement is then set as the new reference cardiac output.

On the other hand, the difference |PCCO−CO_{Ref}| may generally be determined “beat-to-beat” for each PCCO measurement, though longer time intervals are also possible. If “outliers”, that is to say (isolated) parameter values that, are determined to deviate substantially (in the same direction) from both the preceding and the following parameter value, are to be prevented from triggering a recalibration, a modified calibration criterion may be used. For example, it may be defined as a (possibly additional) necessary condition for triggering the calibration signal that the difference |PCCO−CO_{Ref}| must be greater than the tolerance value for multiple, for example three or five, consecutive derived parameter values, or that the difference |PCCO−CO_{Ref}| must exceed the tolerance value in a minimum percentage of the PCCO values determined within a defined period of time (for example |PCCO−CO_{Ref}|>Tol for at least 50% of the values derived in the last minute). Alternatively, instead of the difference |PCCO−CO_{Ref}| the deviation of the arithmetical, mean of the last i parameter values is used for the calibration criterion, such that a calibration criterion will be triggered when

|(PCCOₘ+PCCOₘ₊₁₊ . . . PCCOₘ₊ᵢ)/i) −CO_{Ref}|>Tol

Instead of calibration criterion

|PCCO−CO_{Ref}|>Tol

is met.

A corresponding filtering of “outliers” by (optionally also weighted) averaging or the requirement for a minimum percentage of values outside the tolerance range within a predefined interval may also be advantageous in an analysis based on the comparison of n-tuples with n>1.

The risk of triggering a calibration due to isolated deviating parameter values may also be reduced by providing a minimum temporal interval between two consecutive calibration signal triggers. A minimum temporal interval between two consecutive calibration signal triggers may also be advantageous to implement for other reasons, for example in order to avoid an additional burden on the patient and/or the medical staff due to frequent-application of an invasive measuring procedure.

Accordingly, the procedure outlined in FIG. 3 may be expanded to a two-stage decision procedure, wherein the query whether |PCCO−CO_{Ref}|>Tol or |[PCCOₘ+PCCOₘ₊₁₊ . . . PCCOₘ₊ᵢ)/i]−CO_{Ref}|>Tol is followed by another query, whether a time that has passed since the last calibration exceeds a predefined period of, for example, 20 minutes, and that the calibration signal will not be triggered until such period has elapsed.

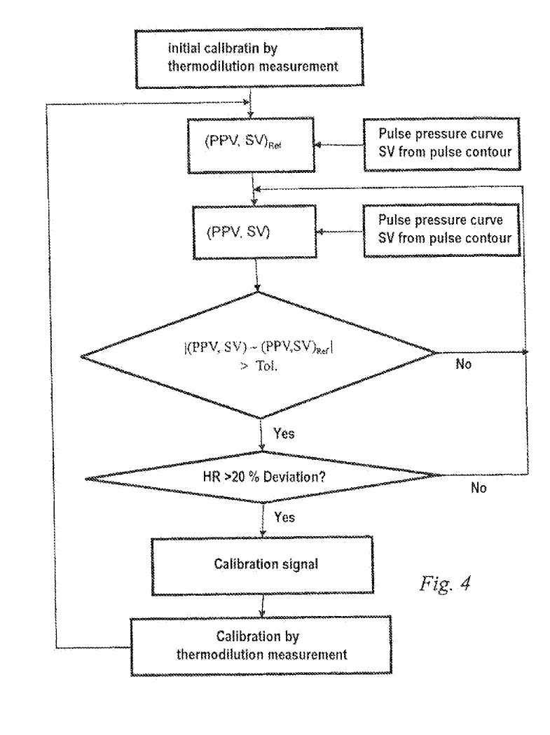

FIG. 4 shows a flow diagram of the initiation of a recalibration according to the invention using the example of a 2-tuple with the pulse pressure variation (PPV) and stroke volume (SV) as constituent components. The triggering means use known algorithms to determine the stroke volume SV and pulse pressure variation PPV, as well as the one or more other haemodynamic parameters of interest at a first point in time. The reference tuple (PPV, SV) Ref obtained in this way is then correlated with the respective one currently derived (PPV, SV) by the triggering means, for example by forming the difference and comparison with a threshold value (tolerance value “Tol”). If the difference |(PPV, SV)−(PPV, SV)_{Ref}|, which may also be interpreted as a distance in the two-dimensional vector space, is smaller than the predefined threshold value, the next parameter is determined. If it is larger, the triggering means queries another criterion, for example deviation in heart rate. If this deviation is greater than a given value, for example >20% of a value determined immediately after a calibration, the calibration signal is triggered by the triggering means, which is followed by the acoustic and/or visual display of the calibration signal and/or an automatic calibration by thermodilution follows. The tuple (PPV; SV) determined immediately before the calibration now serves again as the reference tuple for the subsequent determinations.

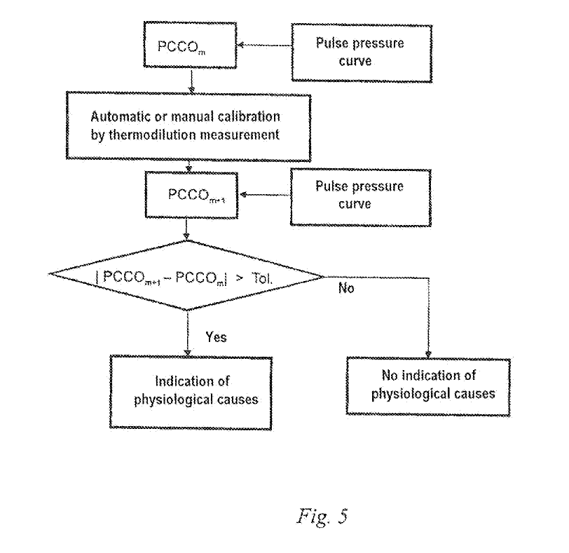

FIG. 5 shows a flow diagram for the evaluation of the calibration result according to a preferred embodiment of the invention. In the evaluation, one or more values of one or more haemodynamic parameters determined immediately before and immediately after a manual or automatic calibration are correlated. In the example shown, a simple comparison of the PCCO collected before and after a calibration is shown. If the two values PCCOₘ₊₁−PCCOₘ differ from, one another by less than a predefined tolerance value “Tol”, this is shown to the user on display 18. Thus the user is made aware that the parameter change in the interval between the last two calibrations is evidently not attributable to artefacts of the algorithm used, a flatline drift or similar, but its causes must rather be physiological.

1. A device for haemodynamic monitoring, wherein the device comprises the following:
reading in means for repeated reading in of data representing at least one physical variable,
calculation means for calculating at least one parameter from the read-in data,
calibrating means for calibrating the device, and
triggering means for triggering a calibration signal depending on the change over time of at least one of the the following:
the read-in data
at least one of the parameters.

2. The device according to claim 1, wherein the triggering means are designed to form a relation between at least one n-tuple with n components determined at a first point in time and at least one n-tuple with n components determined at least one later point in time via a univariate or multivariate analysis procedure, wherein n is a natural number equal to or greater than 1, and the components comprise at least one of the following:
a value of the read-in data
at least one of the parameters.

3. The device according to claim 1, wherein the triggering means are designed to execute at least one mathematical classification method.

4. The device according to claim 1, wherein the triggering means are designed to perform a linear discriminant analysis.

5. The device according to claim 1, wherein the n components comprise at least one parameter selected from the group of heart rate (HR), pulse duration (T), stroke volume (SV), mean arterial pressure (MAP), pulse contour cardiac output (PCCO), thermodilution cardiac output (CO_{TD}), stroke volume variation (SW), systemic vascular resistance (SVR), pulse pressure variation (PPV), fluid responsiveness index (FRI), ventricular contractility (for example dPmx), atrial pressures, ventricular pressures, EKG section data, EKG intervals, characteristics of the medication that affects haemodynamics and respiratory parameters that affect haemodynamics.

6. The device according to claim 2, wherein the n components comprise at least one value of read-in data selected from the group of arterial pressure (P), central venous pressure (CVP), blood temperature (Tb), peripheral oxygen saturation (SpO2), central venous oxygen saturation (ScvO2).

7. The device according to claim 2, wherein the temporal interval between two consecutive determinations of the n-tuple is not more than 1 hour, preferably not more than 15 minutes.

8. The device according to claim 7, wherein the temporal interval between two consecutive determinations of the n-tuple is predefined as a predefined number of the patient's heartbeats.

9. The device according to claim 1, wherein the triggering means are designed to take into account consecutive changes over time, which occur within a predetermined interval, in the respective other direction, of at least one of the following:
the read-in data
at least one of the parameters.

10. The device according to claim 1, which comprises means for indicating the triggering of the calibration signal in at least one of a visual and an acoustic manner.

11. The device according to claim 1, wherein the calibration signal is transmitted to the calibration means for initiating an automatic calibration or recalibration.

12. The device according to claim 1, wherein the calibration comprises a thermodilution measurement.

13. The device according to claim 1, wherein the triggering means are configured to perform a two- or multistage check of the dependency of the change over time in at least one of the following:
the read-in data,
at least one of the parameters,
and the two- or multistage check comprises the hierarchical check of at least two criteria that cause the triggering of the calibration signal and are dependent on the change over time in at least one of the following:
the read-in data,
at least one of the parameters.

14. The device according to claim 1, wherein the device further comprises evaluation means for evaluating at least one of the following:
an arithmetical relation between at least one value; that is read in immediately before a calibration and at least one value that is determined immediately after this calibration,
an arithmetical relation between at least one parameter that is determined immediately before and at least one parameter that is determined immediately after this calibration.

15. The device according to claim 1, wherein the device further comprises input means for inputting at least one of the following:
biometric information about the patient,
categorising information about the patient.

16. The device according to claim 1, which has adaptation means for adapting a criterion that causes the triggering of the calibration signal depending on the change over time of at least one of the following:
the read-in data,
at least one of the parameters, according to at least one of the fallowing:
the input biometric information about the patient, and
the input categorising information about the patient.

17. A method for calibrating a device for haemodynamic monitoring of a patient, comprising:
(a) reading in of data representing at least one physical variable,
(b) calculating at least, one parameter from the read-in data,
(c) triggering a calibration signal depending on the change over time of at least one of the following:
the read-in data, and
the at least one parameter,
(d) calibrating the device in response to the calibration signal.

18. The method according to claim 17 further comprising: evaluating at least one of the the following:
an arithmetical relation between at least one value of the read-in data determined immediately before and at least one value determined immediately following this calibration and,
an arithmetical relation between at least one of the parameters determined immediately before and immediately following this calibration.
